(19) United States
(12) Patent Application Publication
(10) Pub. No.: US 2006/0191571 A1
(43) Pub. Date: Aug. 31, 2006

(54) Fluid concentration sensing arrangement

(76) Inventors: David R. Kattler, Mantua, OH (US); Ronnie A. Browne, Streetsboro, OH (US); Scott Proper, Stow, OH (US)

Correspondence Address: CALFEE HALTER & GRISWOLD, LLP, 800 SUPERIOR AVENUE, SUITE 1400, CLEVELAND, OH, 44114, US

(21) Appl. No.: 11/351,029

(22) Filed: Feb. 9, 2006

Related U.S. Application Data
(60) Provisional application No. 60/652,083, filed on Feb. 11, 2005.
(60) Provisional application No. 60/652,650, filed on Feb. 14, 2005.
(60) Provisional application No. 60/748,817, filed on Dec. 7, 2005.

(51) Int. Cl. G05D 11/00; G01N 33/00

(52) U.S. Cl. 137/93; 73/61.41

(57) ABSTRACT

Fluid flow arrangements that include optical fluid concentration sensors are disclosed. One arrangement directs fluid flow toward or against a sensor window. One arrangement inhibits light from entering a region that is sensed by the sensor. One arrangement includes a plurality of sensors that monitor blended fluids.

### RELATED APPLICATIONS

This application claims the benefit of U.S. Provisional patent application Ser. No. 60/652,083 filed on Feb. 11, 2005 for ARRANGEMENT FOR FLUID CONCENTRATION SENSOR, U.S. Provisional patent application Ser. No. 60/652,650 filed on Feb. 14, 2005 for ARRANGEMENT FOR FLUID CONCENTRATION SENSOR and U.S. Provisional patent application Ser. No. 60/748,817 filed on Dec. 7, 2005 for FLUID CONCENTRATION SENSING ARRANGEMENT, the entire disclosures of which are fully incorporated herein by reference.

### FIELD OF THE INVENTION

The present invention relates to fluid concentration sensing arrangements. More particularly, the invention relates to fluid concentration sensing arrangements that include optical fluid concentration sensors.

### BACKGROUND OF THE INVENTION

Many industrial and manufacturing processes use fluids (i.e. liquids and gasses) to process materials. These fluids are often mixtures or solutions of two or more fluids. The success or failure of processes performed by applying fluids depends on the solution or mixture having the proper concentration of fluids. Measuring these concentrations in an accurate and efficient manner can lead to successful industrial and manufacturing processes.

Industrial and manufacturing processes often depend on bringing components into contact with a fluid or a fluid solution. Examples of such processes are deposition of a solution onto components to create a controlled chemical reaction and washing or rinsing components in a fluid stream to remove contaminates or to stop a chemical reaction. These processes often need fluid flow systems to direct the fluids or solutions to certain locations within the process.

### SUMMARY

In accordance with one aspect of the application, a fluid concentration sensing arrangement is provided that includes a flow member that directs fluid flow toward or against a sensing surface of a fluid concentration sensor. As a result, fluid is constantly against the sensing surface and boundary conditions that occur when fluid travels in a direction that is parallel to a surface are reduced or eliminated. In one embodiment, the flow member includes a generally bowl shaped cavity that directs fluid flow toward or against the sensing surface.

In accordance with another aspect of the application, a fluid concentration sensing arrangement is provided with an opaque material positioned to inhibit light from entering a sensing area. By inhibiting light from entering the sensing area, fluid concentration can be measured more accurately.

One aspect of the application relates to a fluid blending system. One fluid blending system includes a manifold member, a first fluid control valve, first fluid concentration sensor, a second fluid control valve, a second fluid concentration sensor, and a mixed fluid concentration sensor. The first and second valves may be operated based on input from the fluid concentration sensors to control the concentrations of blended fluids.

Another aspect of the present application relates to fixing a window, such as a sapphire, sapphire crystal, glass, quartz, or optical quality plastic window, to a fluid concentration sensor. Eliminating float or relative movement between the window and the fluid concentration sensor can result in more accurate fluid concentration measurements.

Further advantages and benefits will become apparent to those skilled in the art after considering the following description and appended claims in conjunction with the accompanying drawings.

### BRIEF DESCRIPTION OF THE DRAWINGS

FIG. 1 is a perspective view of a fluid concentration sensing arrangement;

FIG. 2 is a sectional view taken along the plane indicated by lines 2-2 in FIG. 1;

FIG. 3 is an exploded perspective view of a fluid concentration sensing arrangement;

FIG. 4 is a perspective view of a fluid concentration sensing arrangement;

FIG. 5 is a sectional view taken along the plane indicated by lines 5-5 in FIG. 4;
FIG. 5A is an enlarged portion of FIG. 5A;

FIG. 6 is an exploded perspective view of a fluid concentration sensing arrangement;

FIG. 7 is an illustration of fluid flow through a flow member of a fluid concentration sensing arrangement;

FIG. 8 is an illustration of fluid flow through a flow member of a fluid concentration sensing arrangement;

FIG. 9 is a perspective view of a fluid concentration sensing arrangement;

FIG. 10 is an elevational view of a fluid concentration sensing arrangement;

FIG. 11 is an elevational view of a fluid concentration sensing arrangement;

FIG. 12 is a sectional view taken along the plane indicated by lines 12-12 in FIG. 10;

FIG. 13 is a sectional view taken along the plane indicated by lines 13-13 in FIG. 11;

FIG. 14 is an elevational view of a fluid concentration sensing arrangement and an attached conduit;

FIG. 15 is an elevational view of a fluid concentration sensing arrangement and an attached conduit;

FIG. 16 is a schematic illustration of a fluid blending system;

FIG. 17 is a top plan view of a fluid blending system;

FIG. 18 is a view taken along lines 18-18 in FIG. 17;

FIG. 19 is a view taken along lines 19-19 in FIG. 18;

FIG. 20 is a sectional view taken along the plane indicated by lines 20-20 in FIG. 19;

FIG. 21 is a schematic illustration of a flow path of the fluid blending system illustrated by FIG. 17;

FIG. 22 is a top plan view of a fluid blending system;

FIG. 23 is a sectional view of a valve shown in FIG. 22 taken along the plane indicated by lines 23-23 in FIG. 22;

FIG. 24 is a sectional view of a fluid concentration sensing arrangement shown in FIG. 22 taken along the plane indicated by lines 24-24 in FIG. 22;

FIG. 25 is a schematic illustration of a flow path of the fluid blending system illustrated by FIG. 22; and

FIG. 26 is a schematic illustration of a fluid purity sensing arrangement.

### DETAILED DESCRIPTION

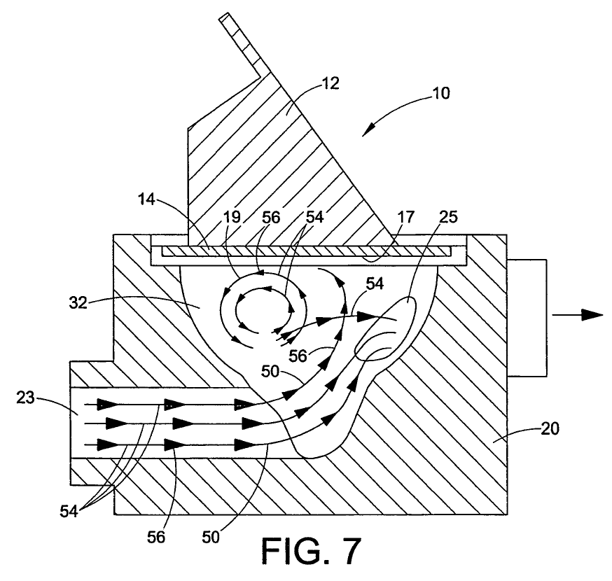

The present invention relates to fluid concentration sensing arrangements 10 that include fluid concentration sensors 12. The illustrated fluid concentration sensors 12 are optical fluid concentrations sensors, but it should be readily apparent that any type of fluid concentration sensor may benefit from features of the disclosed fluid concentration sensing arrangements. One type of optical sensor that may be used is an index of refraction sensor, such as TI refractive index sensor model number TSPR2KXY-R. The disclosed fluid concentration sensing arrangements 10 include a flow member 20 and a fluid concentration sensor 12. The fluid concentration sensor 12 is assembled with the flow member 20, such that a sensing surface 17 of the sensor is in communication with the fluid 19 (see FIG. 7). The fluid may be a liquid or a gas.

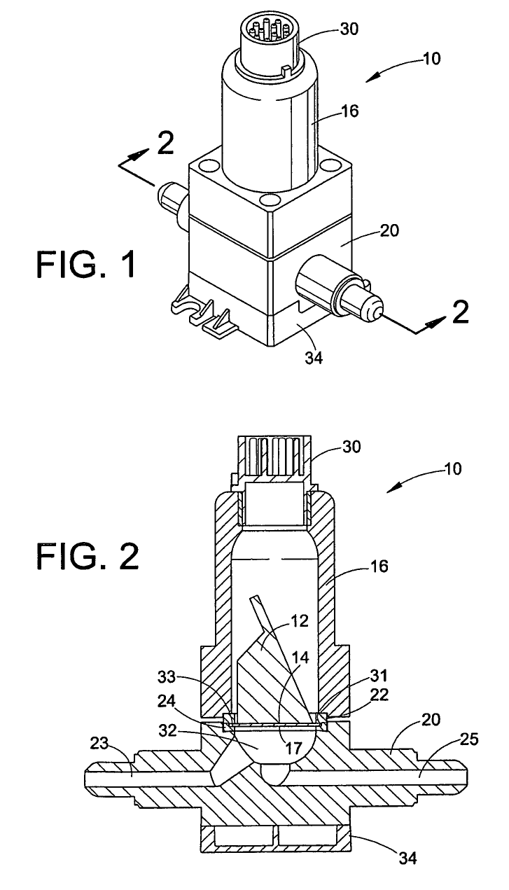

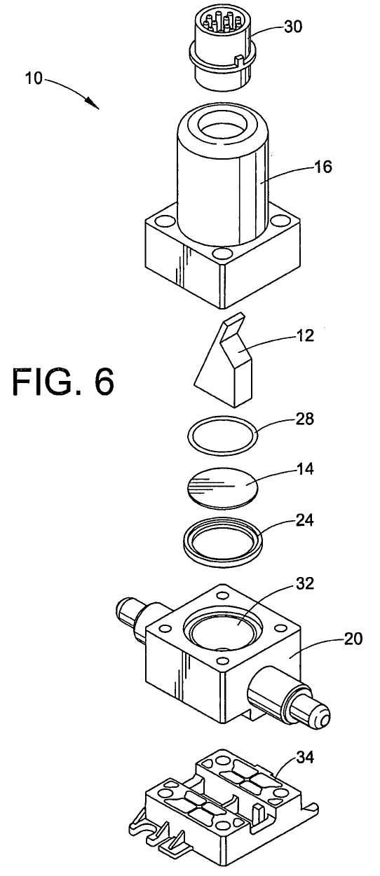

The fluid concentration sensor 12 may be assembled with the flow member 20 in a variety of different ways. FIGS. 1-3 and FIGS. 4-6 illustrate two exemplary mounting arrangements. The illustrated mounting arrangements are examples of the wide variety of mounting arrangements that could be used. Any mounting arrangement that places the fluid concentration sensing surface 17 proximate to the fluid can be employed. In the examples illustrated by FIGS. 1-3 and FIGS. 4-6, an optical liquid concentration sensor 12 is positioned to sense fluid through a window 14, which is a sapphire crystal lens in the exemplary embodiment. The window 14 can be made from a wide variety of different materials. The window can be made from any material that facilitates index of refraction sensing. For example, the window 14 can be made from sapphire, sapphire crystal, quartz, optical lens quality plastics, any crystal material or any material that is suitable for the application. Various criteria may be used to select an appropriate sensor window material. These factors include, but are not limited to, how inert the window material is to the fluids the window will be exposed to, the cost of the window material, and/or the optical performance of the window material. In one embodiment, the window comprises a glass layer and a sapphire layer bonded to the glass layer. For example, a stock fluid concentration sensor may normally be provided with a glass sensing window. To allow the sensor to be used in more environments, a more chemically inert window, such as a sapphire window, may be bonded to the glass window. In another embodiment, a more chemically inert window, such as a sapphire window may be assembled directly with the fluid concentrations sensor, without the glass window. For example, a sapphire window may be bonded to potting material of the fluid concentration sensor. The potting material may be a polycarbonate material.

The window 14 defines the sensing surface 17 that is exposed to the fluid. The window 14 may be fixed to the liquid concentration sensor 12. Fixing the window to the concentration sensor eliminates float of the window with respect to the sensor. As a result, measurement errors caused by movement of the window or lens 14 are eliminated. The window 14 may be fixed to the sensor in a wide variety of different ways. For example, an adhesive may be used to fix the window to the sensor. Acceptable adhesives include epoxies, such as a UV curable optical grade epoxy. One acceptable epoxy is HYSOL OS1102, which can be used to bond a sapphire layer to a glass layer. In one embodiment, and entire interface between the window 14 and the sensor 12 is covered with an adhesive.

The sensor 12 and attached window 14 is placed in a housing 16. In one embodiment, the volume between the housing 16 and the sensor 12 is filled with a potting material. A wide variety of different potting materials may be used. For example, a variety of available dielectric, thermally conductive potting materials may be used. Examples of dielectric, thermally conductive potting materials include urethane dielectric potting materials available from Loctite Corporation. The housing 16 is coupled to a flow member 20. The illustrated flow member 20 defines an inlet opening 23, an outlet opening 25, and sensing cavity 32 between the inlet opening and the outlet opening. The housing 16 may be coupled in a manner that exposes the window 14 to the cavity, and thus allows the sensor 12 to sense the fluid 19 in the cavity.

In many applications, it is beneficial to prevent fluid from entering the housing 16 to protect internal components such as the sensor 12. One method of preventing fluid flow into the housing is to create a seal at the junction between the housing 16 and the window 14 to inhibit the fluid stream from entering the housing 16. In an exemplary embodiment, the coupling between the housing 16 and the flow member 20 is configured such that the majority of the force coupling the housing 16 to the flow member 20 is applied to the housing 16 and the flow member 20 and a small portion of the force is applied to the window 14. The force applied to the window 14 does not damage the window 14, yet is sufficient to provide a reliable seal between the window 14 and the valve body 20.

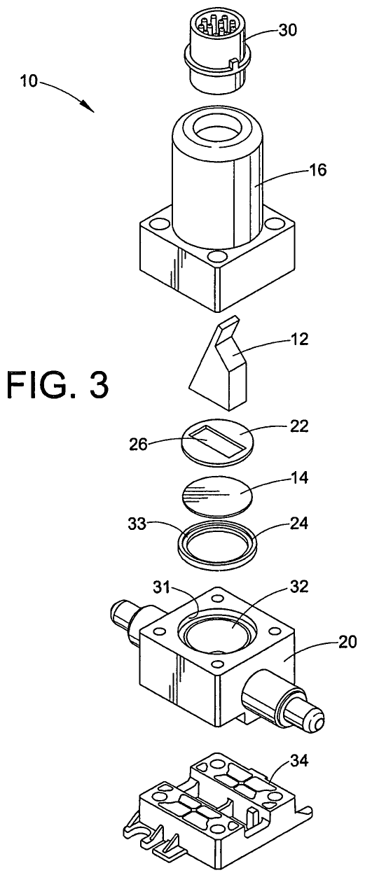

In the example illustrated by FIGS. 2 and 3, a housing interface member 22 and a flow component interface member 24 hold the window 14 in the proper location and alignment. The housing interface member 22 includes a slot 26 into which the sensor 12 is positioned. The flow component interface member is a ring which is dimensioned to fit into a recess 31 of the flow member and has a recess 33 that accepts the window 14. The height of the recess may be slightly smaller than the thickness of the window 14. This difference results in a force being applied to the window to help form the seal between the flow component interface member and the window. The majority of the coupling force securing the housing 16 to the valve body 20 is transferred through the housing interface member 22 and the flow component interface member 24 with a minority of the coupling force transferred through the window 14. The amount of force transferred through the window can be adjusted by changing the depth of the recess and the materials the interface members are made from. The interface members 22, 24 can be any material that allow for a seal to be created and force to be transferred. The material may be, for example, polytetrafluoroethylene (PTFE), also commonly known as teflon.

In one embodiment, a layer of protective material can be placed between the window 14 and the cavity. This material can be any transparent or semi-transparent material, such as teflon. The layer of protective material protects the window 14 from potentially caustic chemicals, may enhance the seal created by the interface members 22, 24, and can allow for a smaller force to be applied to the window 14 to create a seal.

The flow member 20 may be coupled to a base 34. The base 34 allows the fluid concentration sensing arrangement 10 to be conventionally and conveniently secured to a location within the fluid flow system.

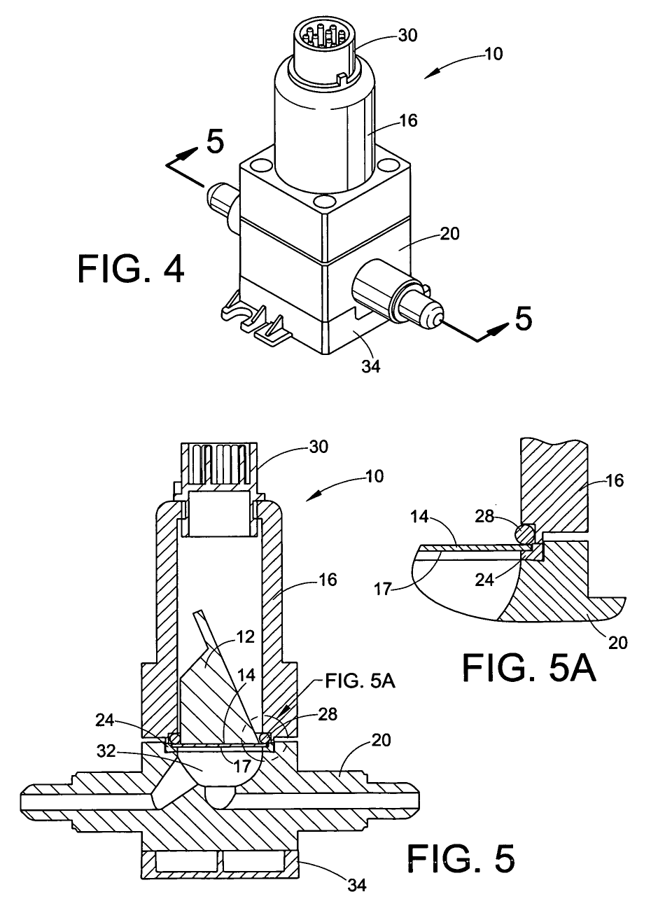

A second example of a mounting arrangement is shown in FIGS. 4-6. An o-ring 28, transfers force from the housing 16 to the window 14 to press the window against the interface member 24 and create a seal there between. The interface member 24 is pressed by the window 14 and the housing 16 against the flow member to create a seal between the flow member and the interface member (See FIG. 5A). A majority of the force coupling the housing 16 and the flow member 20 is transferred directly from the housing 16 to the interface member 24. In the example, the housing 16 defines an annular ring that engages the interface member. A smaller portion of the force is transferred through the o-ring 28. The dimensions and materials of the annular ring, the interface member 24, and the o-ring 28 can be altered to set the amount of force that is transferred through the o-ring and the window. The o-ring 28 is a resilient member which absorbs force and protects the window or lens 14.

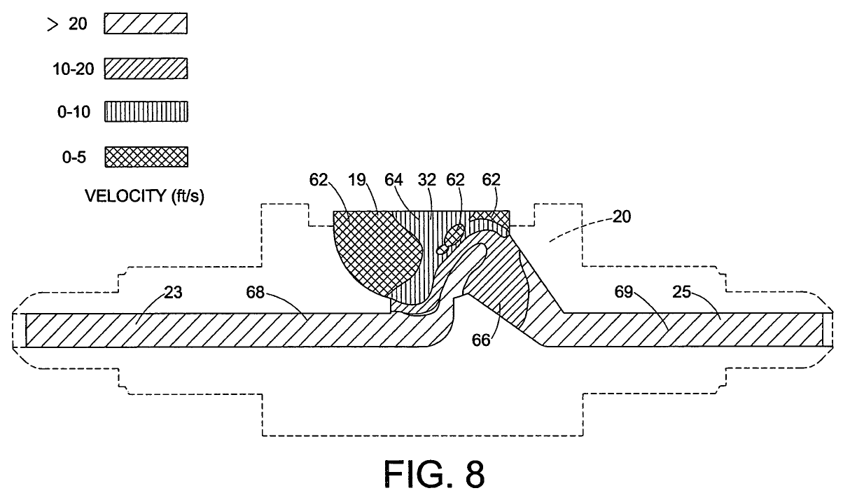

Referring to FIGS. 7 and 8, one aspect of the present application relates to directing fluid 50 flow toward or against a sensing surface 17 of a fluid concentration sensor 12. As a result, fluid 50 is constantly against the sensing surface 17 and boundary conditions, which could inhibit constant contact with a sensing surface, that occur when fluid travels in a direction that is parallel to a surface are reduced or eliminated. In the examples illustrated by FIGS. 7 and 8, the flow member 20 includes an inlet passage 23, an outlet passage 25, and a generally bowl shaped cavity 32 between the inlet and outlet passages that directs fluid flow toward or against the sensing surface. In an exemplary embodiment, a portion of the fluid is diverted toward the sensing surface 17 in a direction that is generally transverse to the sensing surface. The bowl shaped cavity 32 illustrated by FIGS. 7-9 is but one example of the wide variety of different cavity shapes that may be employed. Virtually any cavity shape that directs fluid flow toward or against the sensing surface, instead of parallel to the sensing surface, may be used. In an exemplary embodiment, the sensor 12 measures a concentration of the fluid directed toward the sensing surface 17.

FIG. 7 is a schematic illustration of a flow pattern in a bowl shaped cavity 32 of a flow member 20. Lines 54 illustrate fluid flow through the flow member 20. Arrows 56 represent the velocity of the fluid flowing through the flow member 20. Larger arrows 56 represent faster fluid flow and smaller arrows represent slower fluid flow. FIG. 7 illustrates that a majority of the fluid 50 flows directly from the inlet 23 to the outlet 25 and the flow of this fluid is relatively fast. A portion 56 of the fluid 50 flows toward the sensing surface 17 in the cavity. This fluid circulates in the cavity and gradually flows out the outlet. The flow of fluid toward the sensing surface 17 and the circulation of the flow in cavity is significantly slower than the flow directly from the inlet 23 to the outlet 25. In the exemplary embodiment, the sensor measures the reflectivity of the slower moving portion of fluid. Measuring slower moving fluid improves the accuracy with which the sensor can measure the concentration of the fluid.

FIG. 8 is another illustration of a flow pattern in a flow member 20 with a bowl shaped cavity 32. Different cross hatch patterns 62, 64, 66, 68 represent different fluid velocity ranges in the flow device. The patterns 62, 64 are located in the bowl cavity 32 in the region where fluid is directed toward the sensing surface 17 as described with reference to FIG. 7. The patterns 62, 64 represent relatively slow velocities. In one example, pattern 62 represents fluid flow velocity range between 0 and 5 feet per second and pattern 64 represents fluid flow range between 5 and 10 feet per second. The patterns 66, 68 represent relatively higher velocities. In the example, pattern 66 represents a fluid flow velocity range between 10 and 20 feet per second and pattern 68 represents fluid flow velocity that is greater than 20 feet per second. In the example illustrated by FIG. 8, the fluid velocities may correspond to an inlet pressure that is less than 100 lbf/in². For example, the inlet pressure may be approximately 80 lbf/in². In one example, flow in the bowl shaped cavity 32 within 5 mm of the sensing surface of the sensor is less than 10 feet per second. In the exemplary embodiment, pressure is maintained in the cavity 32 and fluid is constantly in contact with the sensing surface.

The accuracy of the concentration measurements made by an optical sensor 12 increases as the time a portion of the fluid stream is viewable by the sensor 12 increases and as the velocity of the viewable fluid decreases. Flow members 20 that have deeper cavities 32 or bowls increase the time in which a portion of the fluid stream is viewable by the sensor 12 and decrease the velocity of the fluid viewed by the sensor. As a result, the deep bowl cavity increases the accuracy of the concentrations observed by the sensor 12. Examples of flow members with deep bowl shaped cavities are the valve bodies disclosed by U.S. Pat. No. 6,394,417 to Brown for Sanitary Diaphragm Valve granted May 28, 2002 (herein the '417 patent) and U.S. Pat. No. 6,123,320 to Rasanow for Sanitary Diaphragm Valve granted Sep. 26, 2000 (herein the '320 patent), which are hereby incorporated by reference. The valve bodies disclosed by the '417 patent and the '320 patent may be used as the flow members referred to herein. The deep bowl feature of the valve body increases the time that a portion of the fluid stream is viewable to the sensor 12, since the time it takes time for a portion of the fluid that circulates in the bowl to exit the bowl increases. The deep bowl valves disclosed and incorporated in the references listed above have relatively small footprints. This allows for flexibility in locating fluid concentration assemblies into a fluid flow system.

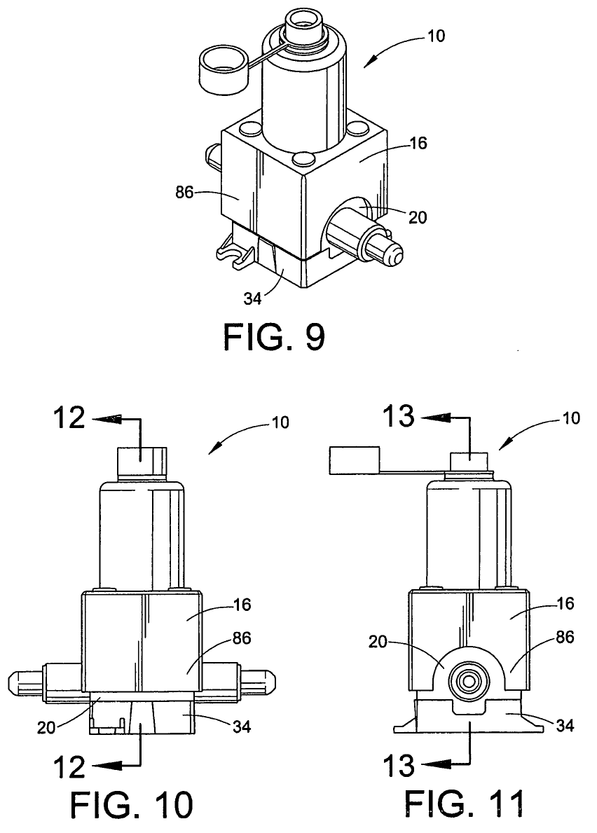

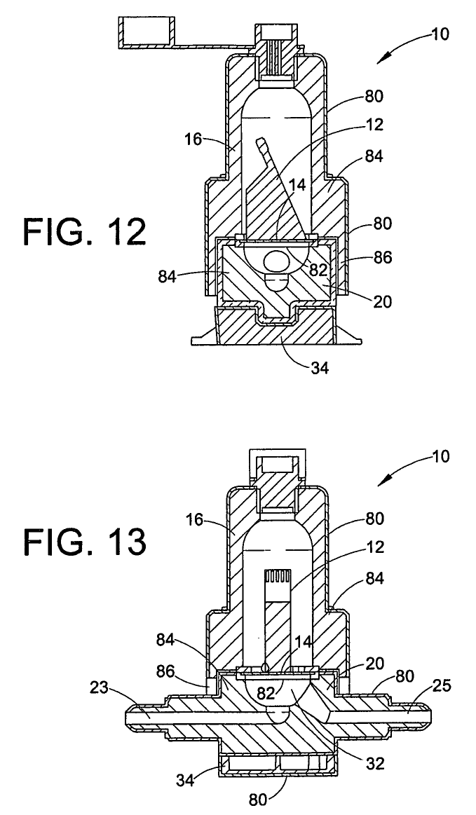

Referring to FIGS. 9-15, another aspect of the present application is a fluid concentration sensing arrangement that is provided with an opaque material 80 positioned to inhibit light from entering a sensing area 82 (FIGS. 12 and 13). FIGS. 12 and 13 illustrate examples of different locations of the flow member 20 and the housing 16 where the opaque material can be positioned. The opaque material 80 may be applied at locations other than the locations illustrated by FIGS. 12 and 13. Further, the opaque material may not be applied at all the locations illustrated in FIGS. 12 and 13 in some embodiments. In one embodiment, a carbon black pigment is added to the flow member to make it opaque. The housing 16 may be made from a polypropylene material. The flow member may be made from a PTFE (Teflon) material. The opaque material can be applied to a surface of the housing 16 and/or the flow member. By inhibiting light from entering the sensing area 82, fluid concentration can be measured more accurately.

In the examples illustrated by FIGS. 9-15, the fluid concentration sensing arrangement 10 includes a flow member 20, a fluid concentration sensor 12, a housing 16, and an opaque material 80 (shown in FIGS. 12-15). In this application, the term opaque material means a material that inhibits light rays that can effect a measurement of the sensor 12 from passing into the sensing area 82. The light rays may or may not be visible by the human eye. FIGS. 9-15 illustrate examples of opaque material applied to the fluid concentration sensing arrangement to inhibit light from entering the sensing area. The examples of FIGS. 9-15 are but a few of the wide variety of different ways the opaque material can be applied. The opaque material can be provided on or in one or more components of the fluid concentration sensing arrangement 10 in any manner and at any location that inhibits light from entering the sensing area. In the examples illustrated by FIGS. 9-15, the flow member 20 may be made from an at least partially translucent material 84 (see FIGS. 12 and 13). The opaque material is positioned to inhibit light that can effect measurements of the sensor 12 from entering the cavity. In the example illustrated by FIGS. 9-13, opaque material 80 is applied to the flow member 20 and the housing 16 or bonnet.

In one embodiment, the opaque material may be applied to only one of the flow member 20 and the housing 16 or bonnet. For example, the housing 16 or bonnet illustrated by FIGS. 9-13 includes a shroud portion 86 with opaque material 80 that surrounds the flow member 20. In this example, the opaque material 80 applied to the shroud portion 86 may eliminate the need to apply the opaque material 80 to the flow member 80. Similarly, opaque material applied to the flow member 20 may eliminate the need to apply opaque material to the housing.

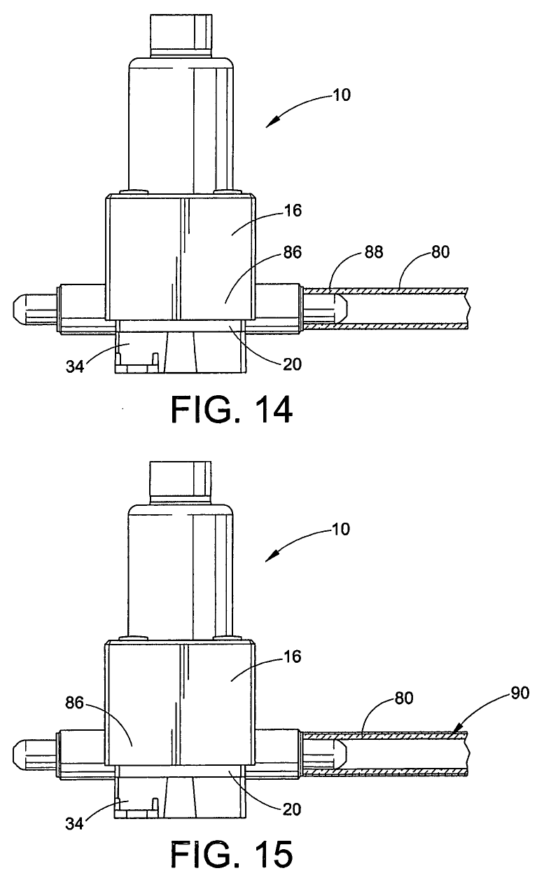

In the example illustrated by FIG. 14, the opaque material 80 comprises an opaque conduit 88 coupled to the inlet 23 or outlet of the flow member 20. The opaque conduit 88 inhibits light from entering the sensing area of the fluid concentration sensing arrangement 10. In the example illustrated by FIG. 15, a conduit 90 is made from an at least partially translucent material and is coupled to the inlet opening. Opaque material 80 is applied to the conduit. The conduit 90 with an opaque coating inhibits light from entering the sensing area of the fluid concentration sensing arrangement 10.

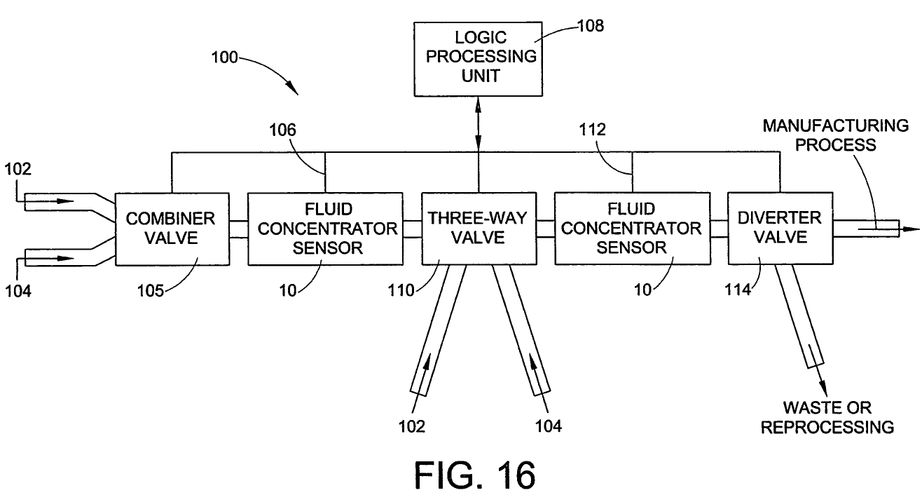

Referring to FIG. 16, another aspect of the present disclosure is the use of fluid concentration sensing assemblies 10 within fluid flow systems 100 to control mixing of fluids. Multiple fluid concentration sensing assemblies 10 may be placed into a fluid flow system to serve a number of functions. For example, multiple fluid concentration sensing assemblies can be placed at different positions in a flow stream to measure the concentration of a fluid solution or blend and be analyzed to correct concentrations that are not within an acceptable ratio. In the example illustrated by FIG. 16, two fluids, 102 and 104, are blended by a combiner valve 105. The fluids may be fluids that are used in any application. For example, the fluids may be used in a industrial or manufacturing process. A fluid concentration sensing assembly 10 is positioned downstream of the combiner valve at position 106 to measure the concentrations of fluid 102 and/or fluid 104. The measurement is relayed to a logic processing unit 108. If the concentration of the blend of fluids 102 and 104 is not within an acceptable range or ratio, the logic processing unit can send a command to a downstream three-way valve 110 that controls access to the fluid stream. This command can instruct the valve to add an appropriate amount of fluid 102 or fluid 104 to the fluid stream to bring the ratio of fluid 102 and fluid 104 into an acceptable range. A second fluid concentration sensing arrangement 10 is placed downstream of the three-way valve at position 112 to again measure the concentrations of fluid 102 and/or fluid 104. The measurement is relayed to the logic processing unit 108 to verify that the fluid stream concentration is correct. If the concentration was not corrected, the logic processing unit can relay a command to a downstream diverter valve to divert 114 or dump the fluid stream from the process path to prevent an error in the manufacturing process.

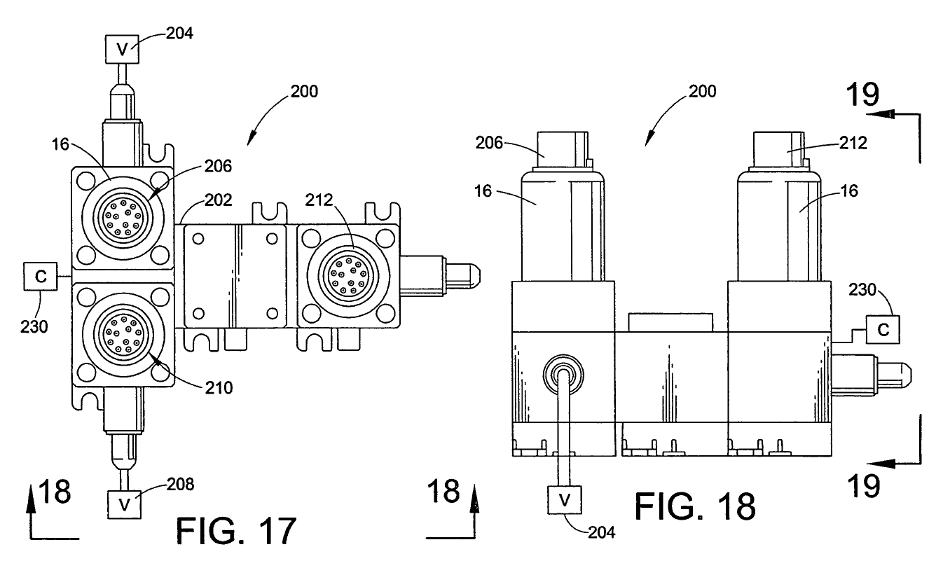

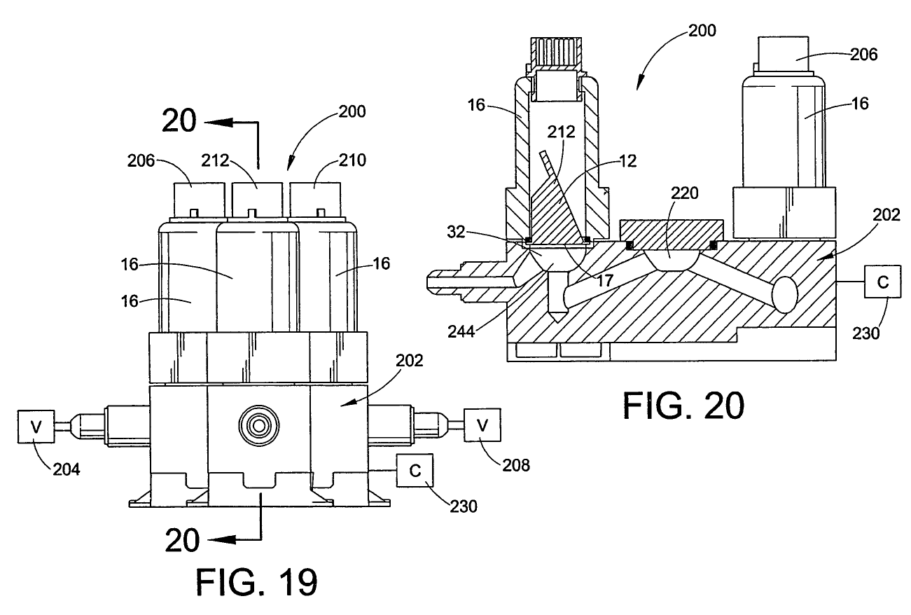

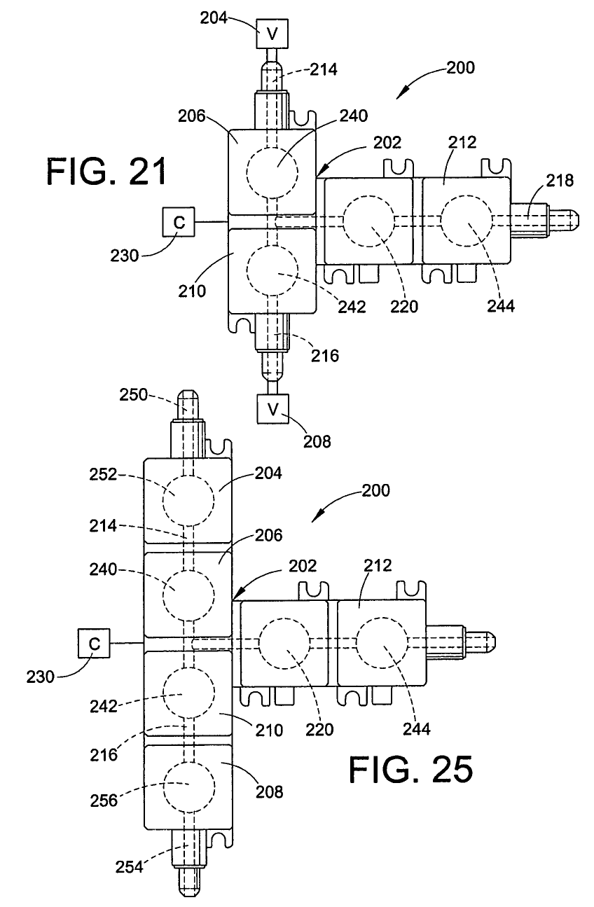

FIGS. 17-21 and 22-25 illustrate two examples of fluid blending systems 200. The fluid blending system 200 illustrated by FIGS. 17-21 includes a manifold member 202, a first fluid control valve 204, a first fluid concentration sensor 206, a second fluid control valve 208, a second fluid concentration sensor 210, and a mixed fluid concentration sensor 212. In the example illustrated by FIGS. 17-21, the control valves 204, 208 are separate from the manifold member 202. FIG. 21 schematically illustrates the flow passages defined by the manifold member 202. The manifold member defines a first fluid inlet passage 214, a second fluid inlet passage 216, a mixed fluid outlet passage 218, and a mixing cavity 220 in fluid communication with the first fluid inlet passage, the second fluid inlet passage and the mixed fluid outlet passage. The first fluid control valve 204 controls flow of a first fluid to the first fluid inlet passage 214. The first fluid concentration sensor 206 measures a concentration of the first fluid flowing through the first fluid inlet passage 214. The second fluid control valve 208 controls flow of a second fluid to the second fluid inlet passage 216. The second fluid concentration sensor 210 measures a concentration of the second fluid flowing through the second fluid inlet passage. The mixed fluid concentration sensor 212 measures a concentration of fluid mixed in the mixing cavity 220. A controller 230 is in communication with the fluid concentration sensors 206, 210, 212 and the valves 204, 208. The controller 230 operates the first fluid control valve 204 and the second fluid control valve 208 based on concentration signals provided by the first fluid concentration sensor 206, the second fluid concentration sensor 210, and the mixed fluid concentration sensor 212. The control valves 204, 208 are controlled to control the concentrations of the first and second fluids in the mixture.

Referring to FIG. 21, the manifold member 202 defines the first inlet passage 214, the second inlet passage 216, a first sensor cavity 240, a second sensor cavity 242, a mixing cavity 220, and a third sensor cavity 244. FIG. 20 illustrates the third sensor cavity 244 and the mixing cavity 220. In an exemplary embodiment, the sensor cavities 240, 242 are substantially the same as cavity 244 and are therefore are not shown in FIG. 20 or described in detail. The sensor cavity 244 is generally bowl shaped. However, the sensor cavity can be any shape that allows fluid concentration to be measured, including shapes that causes fluid to be directed toward or against the sensing surface 17. The illustrated mixing cavity 220 is also illustrated as generally bowl shaped. However, the illustrated mixing cavity can be any shape that is conductive to mixing of fluids that enter the cavity 220. Referring to FIG. 21, the inlet valves 204, 208 are coupled to the first and second inlet passages 214, 216. Fluid concentration sensors 206, 210 are positioned in fluid communication with the first and second sensor cavities 242, 244 (see the exemplary positioning of the sensor 12 in FIG. 20). Referring to FIG. 21, first and second fluids flow from the first and second inlet passages 214, 216 into the first and second sensor cavities 240, 242, where the fluid concentrations sensors 206, 210 measure the concentrations of the first and second fluids. The first and second fluids flow from the first and second sensor cavities 240, 242 into the mixing cavity 220, where the fluids mix. In the illustrated embodiment, separate mixing and third sensor cavities 220, 244 are included. In one embodiment, the third sensor cavity 244 serves as the mixing cavity and the cavity 220 is omitted. One or more fluid concentrations are measured by the mixed fluid sensor 212 at the third sensor cavity 244.

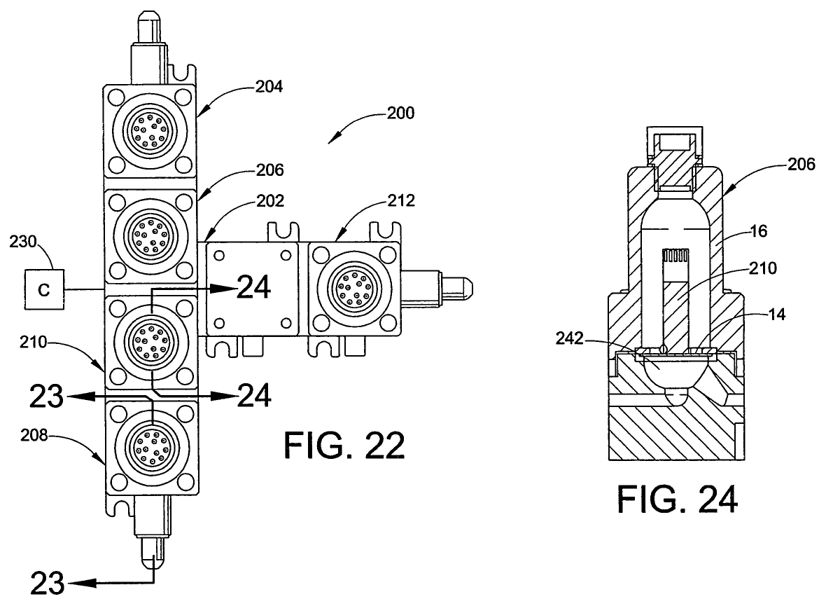

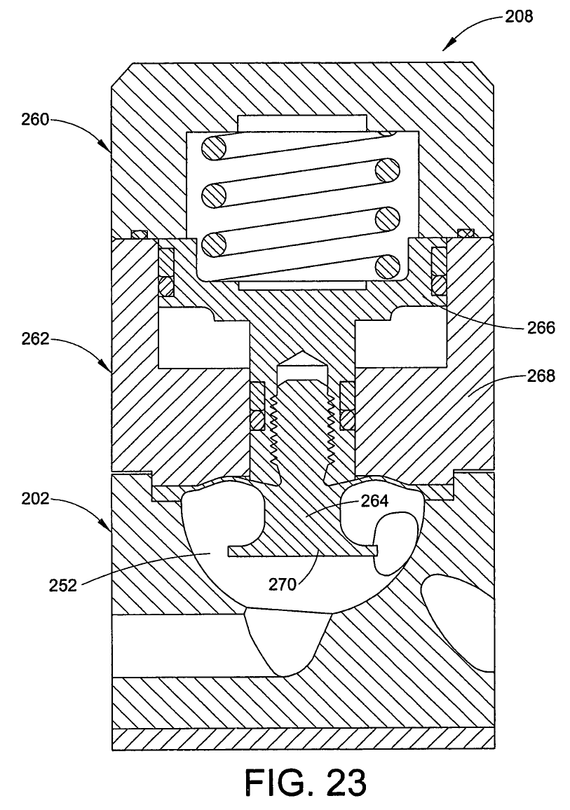

FIGS. 22-25 illustrate an example of a fluid blending system 200 where chambers of the valves 204, 208 are defined by the manifold member 202. Referring to FIG. 25, the manifold member 202 defines a first valve inlet passage 250, a first valve chamber 252, the first inlet passage 214, a second valve inlet passage 254, a second valve chamber 256, the second inlet passage 216, a first sensor cavity 240, a second sensor cavity 242, a mixing cavity 220, and a third sensor cavity 244. The valve inlet passages 250, 254 are in fluid communication with the valve chambers 252, 256. The valve chambers 252, 256 are in fluid communication with the inlet passages 214, 216. Referring to FIG. 23, a sectional view of an exemplary inlet valve 208 is shown. Inlet valve 204 is not described in detail, since inlet valve 204 is substantially the same as inlet valve 208. The inlet valve 208 is defined by the valve chamber 252 defined in the manifold member 202 and a sealing assembly 260 assembled with the manifold member 202. FIG. 23 illustrates one of the wide variety of different sealing assembly and valve cavity arrangements that could be used. In the example, the sealing assembly 260 includes a valve actuator 262 and a diaphragm 264. In this embodiment, the actuator 262 is an air actuator, however any suitable valve actuator may be used. The valve actuator 262 includes an actuator piston 266 that axially moves within an actuator housing 268 to move the diaphragm 264 in the valve chamber 252. The illustrated diaphragm 264 includes a stem tip 270 that opens and closes inlet passage 254 to open and close fluid communication between the valve inlet passage 254 and second concentration sensor inlet passage 216. Further details of valve arrangements that may be adapted for use as the sealing assembly 260 and configurations of valve cavities are disclosed in U.S. Pat. No. 6,394,417 to Browne et al., which is incorporated herein by reference in its entirety. The inlet valves 204, 208, which are integral with the manifold in the example of FIGS. 22-25, selectively allow fluid to flow to the first and second inlet passages 214, 216. Referring to FIG. 24, the fluid concentration sensor 210 is positioned in fluid communication with the first and second sensor cavities 242. Concentration sensors are similarly arranged with respect to cavities 240 and 244. Referring to FIG. 25, first and second fluids flow from the first and second inlet passages 214, 216 into the first and second sensor cavities 240, 242, where the fluid concentrations sensors 206, 210 measure the concentrations of the first and second fluids. The first and second fluids flow from the first and second sensor cavities 240, 242 into a mixing cavity 220, where the fluids mix. In the illustrated embodiment, separate mixing and third sensor cavities 220, 244 are included. In one embodiment, the third sensor cavity 244 serves as the mixing cavity and the cavity 220 is omitted. One or more fluid concentrations are measured by the mixed fluid sensor 212 at the third sensor cavity 244.

In the exemplary embodiment, the sensors 206, 210, 212 are designed for communication with the controller 230. The sensors relay measurement information to the controller, which processes the measurement information and delivers control commands to the valves 204, 208. The examples illustrated by FIGS. 17-25 illustrate blending systems 200 that control blending of two fluids. The blending system 200 can be expanded to control blending of any number of fluids.

The manifold members may be made from a wide variety of different materials. The materials the manifold member is made from may be selected for the application of the blending system. In one embodiment, the manifold member 202 is made from a material that is substantially inert when exposed to cleaning solutions used in the semiconductor industry, for example SC1 (hydrogen peroxide/ammonia aqueous bath) and SC2 (hydrogen peroxide/hydrochloric aqueous bath). Examples of materials that are substantially inert when exposed to many cleaning solutions used in the semiconductor industry include, but are not limited to PTFE (Polytetrafluoroethylene) (Teflon®) or PFA (Perfluoroalkoxy). In an exemplary embodiment, the manifold member is made from a single block or piece of material.

In another exemplary embodiment of the invention, a fluid concentration sensing arrangement may be adapted for detecting an optical characteristic of a fluid used as a refracting medium. One example of such an application is the use of a liquid refracting medium, such as, for example, de-ionized water, between a refractive lens of an optical lithography system and a silicone wafer to be etched by radiation, such as a laser, generated by the optical lithography system. The development of immersion lithography, or the use of a liquid refracting medium in an optical lithography system, more fully described in ICKnowledge.com Technology Backgrounder: Immersion Lithography, has resulted from efforts to improve the resolution of features printed or etched on semiconductor wafers by increasing the index of refraction of the refracting medium. In such an application, the presence of contaminants or impurities in the refracting medium may interfere with the laser etching operation, resulting in errors or inconsistencies in the features etched on the wafers.

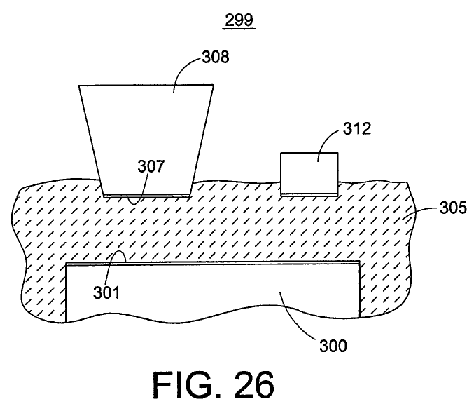

FIG. 26 illustrates an embodiment where an optical sensor 312 is used in immersion lithography to sense an optical property of an immersion liquid 305. FIG. 26 schematically illustrates an example of an immersion lithography arrangement 299. However, the sensor 312 can be used in any immersion lithography arrangement to determine an optical property of the immersion liquid 305. Examples of immersion lithography arrangements are disclosed in “Technology backgrounder: Immersion Lithography,” ICKnowledge.com (2003) and Switckes et al., “Immersion lithography: Beyond the 65 nm node with optics,” Microlithography World, p. 4, (May 2003). In the example illustrated by FIG. 26, a wafer or substrate 300, such as a semiconductor wafer, is immersed in a liquid 305, such as de-ionized water. An etching lens 307 of an optical lithography exposure source 308 is submerged in or in contact with the refracting liquid 305 at a distance from the surface 301 of the substrate 300 to be etched in the illustrated embodiment. The exposure source is adapted to emit a laser, such as a krypton fluoride excimer laser, to etch the substrate surface 301. An optical sensor 312 is likewise submerged in the refracting fluid 305 at a distance from the substrate surface 301 in the illustrated embodiment. The sensor can be placed at any position with respect to the lens and the substrate as long as the sensor is able to sense the optical property of the liquid. The optical sensor 312 may be used to detect an optical characteristic of the liquid 305 related to the purity of the fluid or the presence of contaminants. In one embodiment, the optical sensor 312 is a refractive index sensor, including but not limited to the refractive index sensor 12 described in the above embodiments. The sensor 312 may form part of an index of refraction sensing arrangement, such as any of the index of refraction sensing arrangements 10 described in the above embodiments. The refractive index sensor may be adapted to detect changes over time in the index of refraction of the liquid 305, which may occur as a result of the accumulation of contaminants or impurities in the liquid. The refractive index sensor may also compare the detected index of refraction of the fluid 305 to a predetermined limit value thereby providing notification of a need to clean or replace the refracting fluid 305 before a substrate 300 is improperly etched as a result of refracting fluid impurities.

The arrangement illustrated by FIG. 26 may be used in a method of etching a semiconductor substrate. In the method, the substrate 300 is immersed in a liquid 305. Radiation is emitted through the liquid to etch the surface of the substrate. An optical characteristic of the liquid, that relates to the presence of impurities in the refracting fluid is measured. The measured optical characteristic is compared to a predetermined limit value associated with a limit amount of contamination in the liquid. A signal that the limit amount of contamination has been reached is provided when the predetermined limit value is reached.

It should be understood that the embodiments discussed above are representative of aspects of the invention and are provided as examples and not an exhaustive description of implementations of an aspect of the invention.

While various aspects of the invention are described and illustrated herein as embodied in combination in the exemplary embodiments, these various aspects may be realized in many alternative embodiments, either individually or in various combinations and sub-combinations thereof. Unless expressly excluded herein all such combinations and sub-combinations are intended to be within the scope of the present invention. Still further, while various alternative embodiments as to the various aspects and features of the invention, such as alternative materials, structures, configurations, methods, devices, software, hardware, control logic and so on may be described herein, such descriptions are not intended to be a complete or exhaustive list of available alternative embodiments, whether presently known or later developed. Those skilled in the art may readily adopt one or more of the aspects, concepts or features of the invention into additional embodiments within the scope of the present invention even if such embodiments are not expressly disclosed herein. Additionally, even though some features, concepts or aspects of the invention may be described herein as being a preferred arrangement or method, such description is not intended to suggest that such feature is required or necessary unless expressly so stated. Still further, exemplary or representative values and ranges may be included to assist in understanding the present invention, however, such values and ranges are not to be construed in a limiting sense and are intended to be critical values or ranges only if so expressly stated.

1. A fluid concentration sensing arrangement comprising:
a) a flow member having an inlet opening, an outlet opening and a cavity disposed between the inlet opening and the outlet opening;
b) a fluid concentration sensor assembled with the flow member such that a sensing surface is in communication with the cavity, wherein the cavity directs fluid flow against the sensing surface such that fluid is constantly in contact with the sensing surface.

2. A fluid concentration sensing arrangement comprising:
a) a flow member having an inlet opening, an outlet opening and a generally bowl shaped cavity disposed between the inlet opening and the outlet opening;
b) a fluid concentration sensor assembled with the flow member such that a sensing surface is in communication with the bowl shaped cavity, wherein the bowl shaped cavity directs fluid flow toward the sensing surface.

3. The fluid concentration sensing arrangement of claim 1 wherein the generally bowl shaped cavity directs the fluid flow such that a maximum velocity of the fluid within five millimeters of the sensing surface is less than ten feet per second.

4. The fluid concentration sensing arrangement of claim 1 wherein the generally bowl shaped cavity directs the fluid flow such that a maximum velocity of the fluid within five millimeters of the sensing surface is less than ten feet per second when a pressure at the inlet is less than 100 lbf/in².

5. The fluid concentration sensing arrangement of claim 1 wherein the generally bowl shaped cavity directs fluid flow in a direction that is transverse to the sensing surface.

6. A fluid concentration sensing arrangement comprising:
a) a flow member having an inlet opening, an outlet opening and a cavity disposed between the inlet opening and the outlet opening;
b) a fluid concentration sensor assembled with the flow member such that a sensing surface is in communication with the cavity, wherein the cavity directs fluid flow in a direction that is transverse with respect to the sensing surface, such that a maximum velocity of the fluid within five millimeters of the sensing surface is less than ten feet per second.

7. The fluid concentration sensing arrangement of claim 6 wherein the generally bowl shaped cavity directs the fluid flow such that a maximum velocity of the fluid within five millimeters of the sensing surface is less than ten feet per second when a pressure at the inlet is less than 100 lbf/in².

8. A method of measuring a concentration of a fluid comprising:
a) directing fluid flow with a generally bowl shaped surface toward a sensing surface of a fluid concentration sensor;
b) measuring a concentration of the fluid directed toward the sensing surface by the bowl shaped surface with the fluid concentration sensor.

9. The method of claim 8 wherein a maximum velocity of the fluid near the sensing surface is less than ten feet per second.

10. The method of claim 8 wherein a maximum velocity of the fluid within five millimeters of the sensing surface is less than ten feet per second.

11. The method of claim 8 wherein the maximum velocity of the fluid within five millimeters of the sensing surface is less than ten feet per second when a pressure at an inlet is less than 100 lbf/in².

12. The method of claim 8 wherein the generally bowl shaped surface directs fluid flow in a direction that is transverse to the sensing surface.

13. A method of measuring a concentration of a fluid comprising:
a) directing fluid flow toward a sensing surface of a fluid concentration sensor in a direction that is transverse with respect to the sensing surface, wherein a maximum velocity of the fluid within five millimeters of the sensing surface is less than ten feet per second;
b) measuring a concentration of the fluid directed toward the sensing surface with a fluid concentration sensor.

14. The method of claim 8 wherein the maximum velocity of the fluid within five millimeters of the sensing surface is less than ten feet per second when a pressure at an inlet is less than 100 lbf/in².

15. A fluid concentration sensing arrangement comprising:
a) a flow member made from an at least partially translucent material having an inlet opening, an outlet opening and a cavity disposed between the inlet opening and the outlet opening;
b) a fluid concentration sensor assembled with the flow member such that a sensing surface is in communication with the cavity;
c) an opaque material positioned to inhibit light from entering the cavity.

16. The fluid concentration sensing arrangement of claim 15 wherein the opaque material is applied to the flow member.

17. The fluid concentration sensing arrangement of claim 15 wherein a conduit made from an at least partially translucent material is coupled to the inlet opening and the opaque material is applied to the conduit.

18. The fluid concentration sensing arrangement of claim 15 further comprising a bonnet that covers the fluid concentration sensor, wherein the opaque material is applied to the bonnet.

19. The fluid concentration sensing arrangement of claim 18 wherein the bonnet includes a shroud portion with opaque material that at least partially surrounds the flow member.

20. The fluid concentration sensing arrangement of claim 15 wherein the opaque material comprises an opaque conduit coupled to the inlet opening.

21. A method of measuring a concentration of a fluid comprising:
a) providing fluid flow through an at least partially translucent material to a sensing area of a fluid concentration sensor;
b) inhibiting ambient light from passing through the at least partially translucent material and entering the sensing area;
c) measuring a concentration of the fluid in the sensing area.

22. A fluid blending system comprising:
a) a manifold member defining:
i) a first fluid inlet passage;
ii) a second fluid inlet passage;
iii) a mixed fluid outlet passage;
iv) a mixing cavity in fluid communication with the first fluid inlet passage, the second fluid inlet passage; and the mixed fluid outlet passage;
b) a first fluid control valve assembled with the manifold member for controlling flow of a first fluid to the first fluid inlet passage;
c) a first fluid concentration sensor assembled with the manifold member for measuring a concentration of the first fluid flowing through the first fluid inlet passage;
d) a second fluid control valve assembled with the manifold member for controlling flow of a second fluid to the second fluid inlet passage;
e) a second fluid concentration sensor assembled with the manifold member for measuring a concentration of the second fluid flowing through the second fluid inlet passage;
f) a mixed fluid concentration sensor assembled with the manifold member for measuring a concentration of fluid mixed in the mixing cavity.

23. The fluid blending system of claim 22 further comprising a controller in communication with the first fluid control valve, the second fluid control valve, the first fluid concentration sensor, the second fluid concentration sensor, and the mixed fluid concentration sensor, wherein the controller operates the first fluid control valve and the second fluid control valve based on concentration signals provided by the first fluid concentration sensor and the second fluid concentration sensor.

24. The fluid blending system of claim 22 further comprising a controller in communication with the first fluid control valve, the second fluid control valve, the first fluid concentration sensor, the second fluid concentration sensor, and the mixed fluid concentration sensor, wherein the controller operates the first fluid control valve and the second fluid control valve based on concentration signals provided by the first fluid concentration sensor, the second fluid concentration sensor, and the mixed fluid concentration sensor.

25. The fluid blending system of claim 22 wherein the manifold is constructed from a single block of material.

26. The fluid blending system of claim 22 wherein the manifold is constructed from a single block of material and valve ports of the first fluid control valve are defined in the block.

27. The fluid blending system of claim 22 wherein the manifold is constructed from a single block of material and the first fluid control valve is a diaphragm valve having flow passages defined in the block.

28. The fluid blending system of claim 22 wherein the first fluid is a hydrogen peroxide and ammonia solution and the second fluid is a hydrogen peroxide and hydrochloric solution and the manifold is made from a material that is substantially chemically inert when exposed to the first and second fluids.

29. The fluid blending system of claim 22 wherein the first fluid concentration sensor, the second fluid concentration sensor, and the mixed fluid concentration sensor are optical fluid concentration sensors.

30. The fluid blending system of claim 22 wherein the first fluid concentration sensor, the second fluid concentration sensor, and the mixed fluid concentration sensor measure index of refraction to determine fluid concentration.

31. A method of blending fluids,
a) measuring a concentration of a first fluid;
b) measuring a concentration of a second fluid;
c) mixing the first and second fluids;
d) measuring a concentration of a mixture of the first and second fluids;
e) controlling flow of the first and second fluids to be mixed based on the concentrations of the first fluid, the second fluid and the mixture.

32. The method of claim 31 wherein the first and second fluids are gasses.

33. The method of claim 31 wherein the concentrations of the fluids are measured by measuring an optical property of each fluid.

34. The method of claim 31 wherein the concentrations of the fluids are measured by measuring an index of refraction of each fluid.

35. The method of claim 31 wherein the first fluid is SC1 and the second fluid is SC2.

36. A fluid concentration sensing arrangement comprising:
a) a flow member having an inlet opening, an outlet opening and a cavity disposed between the inlet opening and the outlet opening;
b) a fluid concentration sensor assembled with the flow member
c) a crystal window fixed to the fluid concentration sensor such that the crystal window is in communication with the cavity.

37. The fluid concentration sensing arrangement of claim 36 wherein the crystal window is glued to the fluid concentration sensor.

38. The fluid concentration sensing arrangement of claim 36 wherein the crystal window is fixed to the fluid concentration sensor with a ultraviolet curable sealant.

39. The fluid concentration sensing arrangement of claim 36 wherein the crystal window comprises sapphire.

40. A method of assembling a fluid concentration sensing arrangement comprising:
a) fixing a fluid concentration sensor to a sapphire window;
b) clamping the fluid concentration sensor and sapphire window to a flow member having an inlet opening, an outlet opening and a generally bowl shaped cavity disposed between the inlet opening and the outlet opening, such that the sapphire window is in communication with the bowl shaped cavity.

41. An immersion lithography etching arrangement comprising:
a liquid;
a substrate, immersed in said liquid;
an optical lithography etching lens immersed in said liquid and arranged to etch a pattern in the substrate;
an optical sensor, immersed in the liquid to detect characteristics of the liquid.

42. The etching arrangement of claim 41 wherein the optical sensor is a refractive index sensor.

43. The etching arrangement of claim 41 wherein the optical sensor is configured to detect impurities in the fluid.

44. A method of etching a semiconductor substrate comprising:
immersing the substrate in a liquid;
emitting radiation through the liquid to etch the surface of the substrate;
detecting an optical characteristic of the liquid, wherein the optical characteristic relates to the presence of impurities in the refracting fluid;
comparing the optical characteristic to a predetermined limit value associated with a limit amount of contamination in the liquid;
providing a signal that the limit amount of contamination has been reached when the predetermined limit value is reached.
